(19) United States
(12) Patent Application Publication
(10) Pub. No.: US 2023/0072381 A1
(43) Pub. Date: Mar. 9, 2023

(54) AIR CURTAIN SYSTEM INCLUDING PLANT CULTIVATION DEVICE FOR AIR PURIFICATION

(71) Applicants: Byoung Woo KIM, Seoul (KR); Minji KIM, Seoul (KR); Ki Cheol HAN, Seoul (KR); Hyewon KIM, Seoul (KR); Soo Bin LEE, Seoul (KR); Min Jung KIM, Gyeonggi-do (KR)

(72) Inventor: Byoung Woo KIM, Seoul (KR)

(73) Assignees: Byoung Woo KIM, Seoul (KR); Minji KIM, Seoul (KR); Ki Cheol HAN, Seoul (KR); Hyewon KIM, Seoul (KR); Soo Bin LEE, Seoul (KR); Min Jung KIM, Gyeonggi-do (KR)

(21) Appl. No.: 17/802,144

(22) PCT Filed: Feb. 25, 2021
(86) PCT No.: PCT/KR2021/002357

§ 371 Date: Aug. 25, 2022

(30) Foreign Application Priority Data
Feb. 25, 2020 (KR) 10-2020-0023173

(51) Int. Cl. A01G 31/02; F24F 9/00; F24F 8/175; F24F 8/99

(52) U.S. Cl. CPC A01G 31/02; F24F 9/00; F24F 8/175; F24F 8/99

(57) ABSTRACT

Disclosed herein is an air curtain system including a plant cultivation device for air purification. The air curtain system includes: an air purification wall part forming a vertical body of the air curtain system and having a sidewall on which plants are planted, the air purification wall part drawing in air and filtering out pollutants including fine dust and ultrafine dust from the drawn-in air; an air curtain injection part forming a roof of the air curtain system and having an air discharge port formed along a periphery of the roof, the air curtain injection part receiving the air filtered through the air purification wall part and discharging the received air vertically downwards through the air discharge port; and an air suction part forming a floor of the air curtain system and having an air suction port formed along a periphery of the floor, the air suction part suctioning the air discharged through the air discharge port of the air curtain injection part.

### TECHNICAL FIELD

The present invention relates to an air curtain system including a plant cultivation device and, more particularly, to an air curtain system including a plant cultivation device, which can form an air curtain using purified air while providing air circulation by filtering out fine dust and ultrafine dust from air through an air purification wall part having plants planted thereon, discharging the filtered air through an air curtain injection part, and suctioning the discharged air through an air suction part forming a floor of the air curtain system.

### BACKGROUND ART

In urban areas, plants such as trees and flowers are planted, flowerpots are placed, or parks are created for the purpose of improving air quality and urban aesthetics though improvement in urban environment.

Generally, planting plants requires a planar space in which the plants take root. In order to overcome this spatial limitation, there is increasing demand for vertical gardens allowing plants to grow along vertical surfaces of an inner or outer wall of a building.

Such a vertical garden provides various benefits, including air purification by plants absorbing carbon dioxide from the atmosphere and producing oxygen during photosynthesis, reduction in urban noise by plant leaves refracting and absorbing sound, improvement in urban appearance, and providing stress relief and recovery from fatigue to viewers.

### DISCLOSURE

### Technical Problem

In recent years, the concentration of fine dust and ultrafine dust in the atmosphere has increased sharply, raising concerns about air pollution. Among dust contained in the atmosphere, fine dust refers to particulate matter having a particle diameter of less than 10 μm and ultrafine dust refers to particulate matter having a particle diameter of less than 2.5 μm.

Fine dust is a Group 1 carcinogen designated by the International Agency for Research on Cancer (IARC), which is an agency of the World Health Organization (WHO), in 2013. In Korea, when the concentration of fine dust reaches a severe level, emergency reduction measures, such as limiting vehicle use, are taken to reduce the concentration of fine dust.

Embodiments of the present invention provide an air curtain system including a plant cultivation device for air purification, which can remove pollutants including fine dust from the atmosphere, form an air curtain through circulation of purified air, and improve urban appearance using plants.

### Technical Solution

In accordance with one aspect of the present invention, there is provided an air curtain system including a plant cultivation device for air purification, the air curtain system including: an air purification wall part forming a vertical body of the air curtain system and having a sidewall on which plants are planted, the air purification wall part drawing in air and filtering out pollutants including fine dust and ultrafine dust from the drawn-in air;

an air curtain injection part forming a roof of the air curtain system and having an air discharge port formed along a periphery of the roof, the air curtain injection part receiving the air filtered through the air purification wall part and discharging the received air vertically downwards through the air discharge port; and

an air suction part forming a floor of the air curtain system and having an air suction port formed along a periphery of the floor, the air suction part suctioning the air discharged through the air discharge port of the air curtain injection part.

The air purification wall part may include: a plant wall having a sidewall on which plants are planted; an intake fan disposed inside the sidewall of the plant wall and drawing air into the plant wall; and a fine dust filter disposed on the sidewall of the plant wall and filtering out pollutants including fine dust and ultrafine dust from the air drawn into the plant wall.

The air curtain system may further include: a solar panel disposed on the roof formed by the air curtain injection part; and a storage battery storing and supplying electricity produced by the solar panel.

The air curtain system may further include: an air jet pump receiving air filtered through the air purification wall part and delivering the received air at high pressure to the air discharge port of the air curtain injection part; and an air intake device disposed in the air suction part, the air intake device including a ventilator forcing air descending after forming an air curtain into the air suction port such that the air circulates to the air curtain injection part, and a ventilator motor driving the ventilator, wherein the air jet pump and the ventilator motor are driven by electricity supplied from the storage battery.

The air discharge port may include: a duct through which air to be discharged is transferred, the duct having a circular cross-section and being disposed along the periphery of the roof; an outlet through which air for formation of the air curtain is discharged, the outlet having a narrower cross-section than the duct and extending vertically downwards along the duct; and a protrusion protruding inside an exit of the outlet, the protrusion narrowing a cross-section of the exit of the outlet to increase a flow velocity of the air to be discharged.

The roof formed by the air curtain injection part and the floor formed by the air suction part may have a circular or elliptical shape, such that the air curtain system is provided in the form of a circular cylinder or an elliptical cylinder.

The roof formed by the air curtain injection part and the floor formed by the air suction part may have a rectangular shape, such that the air curtain system is provided in the form of a cuboid.

The air curtain system may further include: a water supply pipe disposed along the sidewall of the plant wall and supplying water to the plants planted on the plant wall; a high-pressure nozzle disposed at an upper end of the water supply pipe and spraying water into the atmosphere to remove pollutants from air; and a high-pressure pump delivering water to the water supply pipe and the high-pressure nozzle, the high-pressure pump being driven by electricity received from the storage battery.

### Advantageous Effects

The present invention provides an air curtain system including a plant cultivation device, which can form an air curtain in a semi-permanent manner using purified air while providing air circulation by filtering out pollutants including fine dust and ultrafine dust from air through an air purification wall part having plants planted thereon, discharging the air with the pollutants filtered out therefrom through an air curtain injection part, and suctioning the discharged air through an air suction part forming the floor of the air curtain system.

The air curtain system according to the present invention can improve air quality by removing carbon dioxide and generating oxygen through photosynthesis of planted plants and effectively removing pollutants from the atmosphere using a fine dust filter and jets of water, can form and circulate a pleasant air curtain using purified air, and can be used semi-permanently by operating each device in the system with electricity produced by a solar panel disposed on the roof of the system.

In addition, the air curtain system according to the present invention can improve urban appearance using planted plants and a rainbow formed by reflection, refraction, and dispersion of sunlight in sprayed water droplets. Accordingly, when the air curtain system is used as a bus stop or a smoking room in downtown areas, the air curtain system can effectively isolate a space inside the system from the outside of the system while improving urban appearance.

### DESCRIPTION OF DRAWINGS

FIG. 1 is a schematic view of an air curtain system including a plant cultivation device for air purification according to one embodiment of the present invention.

FIG. 2 is a schematic view of an air curtain system including a plant cultivation device for air purification according to another embodiment of the present invention.

FIG. 3 is a schematic sectional view of an air purification wall part of the air curtain system of FIG. 1 or FIG. 2.

FIG. 4 is a schematic diagram illustrating a concept of supplying electricity produced by a solar panel on a roof of the air curtain system of FIG. 1 or FIG. 2 to each device in the air curtain system.

FIG. 5 is a schematic cross-sectional view of an air discharge port of an air curtain injection part of the air curtain system of FIG. 1 or FIG. 2.

### MODE FOR INVENTION

The above and other aspects, features, and advantages of the present invention will become apparent from the detailed description of the following embodiments in conjunction with the accompanying drawings.

Hereinafter, embodiments of the present invention will be described with reference to the accompanying drawings. It should be noted that like components will be denoted by like reference numerals throughout the specification and the accompanying drawings.

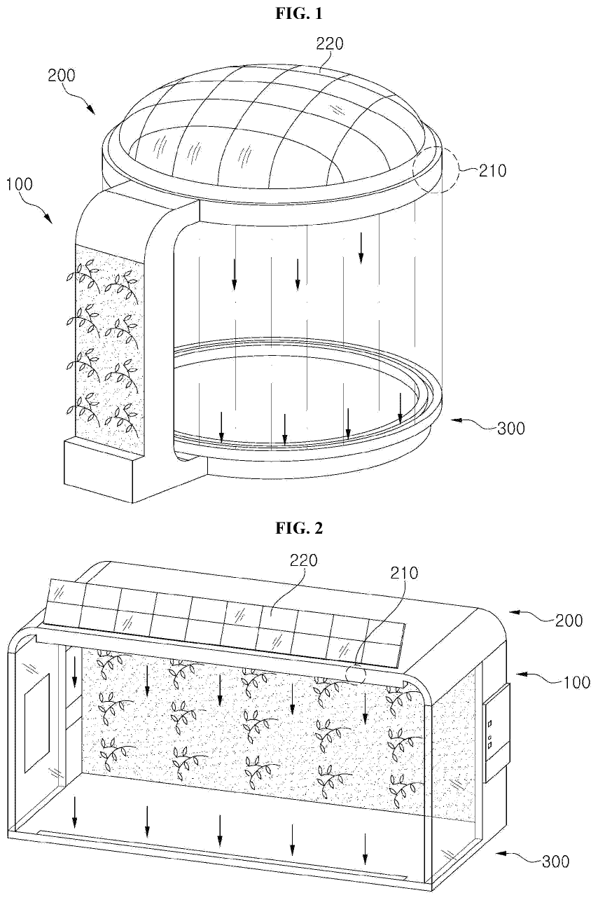

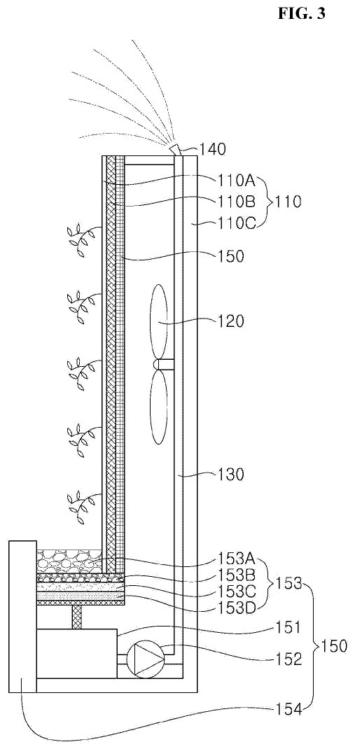

FIG. 1 is a schematic view of an air curtain system including a plant cultivation device for air purification according to one embodiment of the present invention, and FIG. 2 is a schematic view of an air curtain system including a plant cultivation device for air purification according to another embodiment of the present invention.

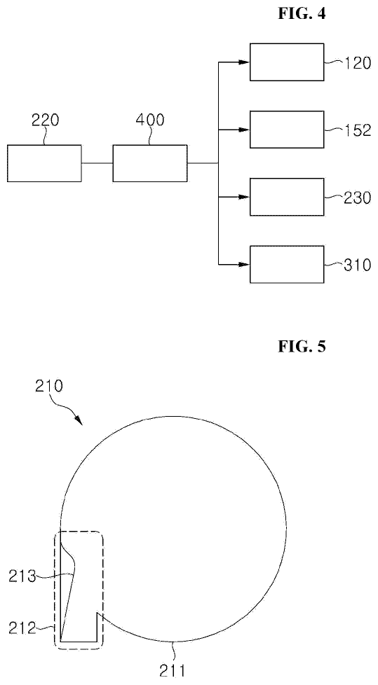

FIG. 3 is a schematic sectional view of an air purification wall part of the air curtain system of FIG. 1 or FIG. 2, and FIG. 4 is a schematic diagram illustrating a concept of supplying electricity produced by a solar panel on a roof of the air curtain system of FIG. 1 or FIG. 2 to each device in the air curtain system. FIG. 5 is a schematic cross-sectional view of an air discharge port of an air curtain injection part of the air curtain system of FIG. 1 or FIG. 2.

Referring to FIG. 1 and FIG. 2, the air curtain system according to the present invention includes: an air purification wall part 100 forming a vertical body of the air curtain system and having a sidewall on which plants are planted, the air purification wall part 100 drawing in air and filtering out pollutants including fine dust and ultrafine dust from in the drawn-in air; an air curtain injection part 200 forming a roof of the air curtain system and having an air discharge port formed along a periphery of the roof, the air curtain injection part 200 receiving the air filtered through the air purification wall part and discharging the received air vertically downwards through the air discharge port; and an air suction part 300 forming a floor of the air curtain system and having an air suction port formed along a periphery of the floor, the air suction part suctioning the air discharged through the air discharge port of the air curtain injection part 200.

Referring to FIG. 1, the roof formed by the air curtain injection part and the floor formed by the air suction part may have a circular or elliptical shape, such that the air curtain system is provided in the form of a circular cylinder or an elliptical cylinder. By way of another example, referring to FIG. 2, the roof formed by the air curtain injection part and the floor formed by the air suction part may have a rectangular shape, such that the air curtain system is provided in the form of a cuboid. In this way, the air curtain system can define a cylindrical, elliptic cylindrical, or cuboidal space that can accommodate users and can be sealed from the outside environment by an air curtain.

Since the air curtain system can define a space sealed from the outside environment, the air curtain system may be used as a downtown bus stop sealed against exhaust gases and fumes, a smoking room installed in a downtown area, and the like. Here, some wall surfaces of the air curtain system of FIG. 1 or FIG. 2 may be formed of glass, tempered glass, or the like to be used as a space for posting advertisements, or may be provided in the form of a glass screen, an LED panel, or the like.

Referring to FIG. 3, the air purification wall part 100 includes a plant wall 110 having a sidewall on which plants are planted, an intake fan 120 disposed inside the sidewall of the plant wall and drawing air into the plant wall, a fine dust filter 115 disposed on the sidewall of the plant wall and filtering out pollutants including fine dust and ultrafine dust from the air drawn into the plant wall by the intake fan, a water supply pipe 130 disposed along the sidewall of the plant wall and supplying water to the plants planted on the plant wall, and a high-pressure nozzle 140 disposed at an upper end of the water supply pipe and spraying water into the atmosphere to remove pollutants from air.

In addition, the air purification wall part 100 includes a water supply unit 150 disposed under the plant wall 110 and supplying water to the water supply pipe and the high-pressure nozzle.

The water supply unit 150 includes a water tank 151 storing water to be supplied to the water supply pipe and the high-pressure nozzle, a high-pressure pump 152 pumping water from the water tank to the water supply pipe and the high-pressure nozzle, a water purification member 153 disposed above the water tank, collecting and purifying falling water, and supplying the purified water to the water tank, and a casing 154 disposed at a lower front of the plant wall and housing the water tank, the high-pressure pump, and the water purification member therein.

The air curtain system may further include a controller (not shown) controlling power supply to control operation of the high-pressure pump, supply of water through the water supply pipe, and spraying of water through the high-pressure nozzle.

The water purification member 153 purifies rainwater, water supplied to the plants through the water supply pipe and having been drained, and water sprayed into the atmosphere through the high-pressure nozzle and having fallen from above and supplies the purified water to the water tank. The water purification member 153 may include a filtering layer formed of a natural material to supply water purified through pollutant filtration to the water tank. For example, the water purification member may be formed by sequentially stacking coarse gravel 153A, sand 153B, charcoal 153C, and a filter 153D, as shown in FIG. 3.

Water purified by the water purification member 153 is stored in the water tank 151 before being pumped to the water supply pipe 130 by the high-pressure pump 152.

The water supply pipe 130 may be provided with multiple water jet orifices (not shown) and a water supply control valve (not shown) to supply water to the plants planted on the plant wall.

Purified water delivered to an upper portion of the water supply pipe can be sprayed into a space above the plant wall through the high-pressure nozzle 140.

Water sprayed through the high-pressure nozzle 140 can purify air by collecting dust and pollutants floating in the air, and can aid in humidity and temperature control in hot and dry weather. In addition, in clear weather, a rainbow can be formed by reflection, refraction, and dispersion of sunlight in water sprayed through the high-pressure nozzle 140, thereby providing aesthetic effects.

The plant wall 110 may include a plant-placing fiber layer 110A on which multiple flowerpots having plants planted therein are placed, a coconut fiber layer 110B disposed on an inner surface of the plant-placing fiber layer 110A, and a vertical wall body 110C to which the plant-placing fiber layer 110A and the coconut fiber layer 110B are attached.

The plant-placing fiber layer 110A may be provided with a hanger (not shown) on which the multiple flowerpots are hung, and may be formed of various natural materials, including felt and eco-friendly plant fibers.

The coconut fiber layer 110B may be provided with a feeder (not shown) supplying nutrients aiding in growth of the plants planted on the plant wall.

The fine dust filter 115 and the intake fan 120 may be disposed between the coconut fiber 110B and the vertical wall body 110C.

The fine dust filter 115 may be attached to the water supply pipe. In particular, in order to effectively filter out pollutant particles such as fine dust and ultrafine dust, the fine dust filter may be formed of nanofibers that have been proven to have filtration efficiency against PM-10 (corresponding to fine dust) and PM-2.5 (corresponding to ultrafine dust). Using such a high-efficiency fine dust filter is the equivalent to using a KF (Korea Filter) mask.

Air drawn into the front of the plant wall by the intake fan is purified through the fine dust filter after passing through the plant-placement fiber and the coconut fiber, is moved to the rear of the vertical wall body of the plant wall, is discharged through the air curtain injection part above the air purification wall part, and is circulated while forming an air curtain.

Referring to FIG. 1 and FIG. 2, the air purification wall part 100 may form a column that connects the air curtain injection part forming the roof to the air suction part forming the floor and supports the air curtain system. Air drawn into the air suction part 400 is moved to the air curtain injection part 300 along an air circulation pipe (not shown) in the air purification wall part.

In another embodiment, air purified through the air purification wall part is transferred to a space inside the air curtain system, is forced to the air curtain injection part by a separate intake fan for formation of an air curtain, is discharged through the air curtain injection part, is drawn into the air suction part, and is circulated along the air circulation pipe.

The air curtain system according to the present invention may be installed outdoors. The air curtain system may further include a solar panel disposed on the roof formed by the air curtain injection part and a storage battery storing electricity produced by the solar panel. Thus, the air curtain system can be used semi-permanently with lower operating costs by producing electricity from sunlight using the solar panel disposed on the roof, storing the produced electricity in the storage battery, and supplying the stored electricity to electricity-driven devices in the system. If water sprayed into the atmosphere through the high-pressure nozzle is directed toward the solar panel, the water can block or refract some portion of sunlight, causing reduction in efficiency of the solar panel. Accordingly, the high-pressure nozzle may be disposed obliquely, for example, at an angle of 45 degrees, in a direction facing away from the air curtain injection part on which the solar panel is disposed, as shown in FIG. 3.

The air curtain system may further include: an air jet pump receiving air filtered through the air purification wall part and delivering the received air at high pressure to the air discharge port of the air curtain injection part; and an air intake device disposed in the air suction part and including a ventilator forcing air descending after forming an air curtain to the air suction port such that the air circulates to the air curtain injection part, and a ventilator motor driving the ventilator.

Referring to FIG. 4, the high-pressure pump 152 delivering water to the water supply pipe and the high-pressure nozzle, the intake fan 120 drawing air into the plant wall, the air jet pump 230 forming an air curtain, and the ventilator motor 310 may be driven by electricity produced by the solar panel 220 and having been received from the storage battery 400. For example, the storage battery, the air jet pump, and the ventilator motor may be disposed inside the air purification wall part.

FIG. 5 is an enlarged cross-sectional view of the air discharge port of the air curtain injection part. Referring to FIG. 5, the air discharge port 210 includes a duct 211 through which air to be discharged is transferred, the duct having a circular cross-section and being disposed along the periphery of the roof, an outlet 212 through which air for formation of an air curtain is discharged, the discharge port having a narrower cross-section than the duct and extending vertically downwards along the duct, and a protrusion 213 protruding inside an exit of the outlet to narrow a cross-section of the exit of the outlet to increase a flow velocity of the air to be discharged.

That is, due to the protrusion protruding inside the exit of the outlet, the cross section of the exit becomes narrower, causing increase in flow velocity of air descending vertically along the protrusion, thereby allowing effective formation of an air curtain and improvement in sealing between the inside and outside of the air curtain.

Although some embodiments have been described, it will be apparent to those skilled in the art that these embodiments are given by way of illustration only, and that various modifications, changes, alterations, and equivalent embodiments can be made without departing from the spirit and scope of the invention.

1. An air curtain system comprising a plant cultivation device for air purification, the air curtain system comprising:
an air purification wall part forming a vertical body of the air curtain system and having a sidewall on which plants are planted, the air purification wall part drawing in air and filtering out pollutants comprising fine dust and ultrafine dust from the drawn-in air;
an air curtain injection part forming a roof of the air curtain system and having an air discharge port formed along a periphery of the roof, the air curtain injection part receiving the air filtered through the air purification wall part and discharging the received air vertically downwards through the air discharge port; and
an air suction part forming a floor of the air curtain system and having an air suction port formed along a periphery of the floor, the air suction part suctioning the air discharged through the air discharge port of the air curtain injection part.

2. The air curtain system according to claim 1, wherein the air purification wall part comprises:
a plant wall having a sidewall on which plants are planted;
an intake fan disposed inside the sidewall of the plant wall and drawing air into the plant wall; and
a fine dust filter disposed on the sidewall of the plant wall and filtering out pollutants comprising fine dust and ultrafine dust from the air drawn into the plant wall.

3. The air curtain system according to claim 2, further comprising:
a solar panel disposed on the roof formed by the air curtain injection part; and
a storage battery storing and supplying electricity produced by the solar panel.

4. The air curtain system according to claim 3, further comprising:
an air jet pump receiving air filtered through the air purification wall part and delivering the received air at high pressure to the air discharge port of the air curtain injection part; and
an air intake device disposed in the air suction part, the air intake device comprising a ventilator forcing air descending after forming an air curtain into the air suction port such that the air circulates to the air curtain injection part, and a ventilator motor driving the ventilator,
wherein the air jet pump and the ventilator motor are driven by electricity supplied from the storage battery.

5. The air curtain system according to claim 4, wherein the air discharge port comprises:
a duct through which air to be discharged is transferred, the duct having a circular cross-section and being disposed along the periphery of the roof;
an outlet through which air for formation of the air curtain is discharged, the outlet having a narrower cross-section than the duct and extending vertically downwards along the duct; and
a protrusion protruding inside an exit of the outlet, the protrusion narrowing a cross-section of the exit of the outlet to increase a flow velocity of the air to be discharged.

6. The air curtain system according to claim 5, wherein the roof formed by the air curtain injection part and the floor formed by the air suction part have a circular or elliptical shape such that the air curtain system is provided in the form of a circular cylinder or an elliptical cylinder.

7. The air curtain system according to claim 5, wherein the roof formed by the air curtain injection part and the floor formed by the air suction part have a rectangular shape such that the air curtain system is provided in the form of a cuboid.

8. The air curtain system according to claim 6, further comprising:
a water supply pipe disposed along the sidewall of the plant wall and supplying water to the plants planted on the plant wall;
a high-pressure nozzle disposed at an upper end of the water supply pipe and spraying water into the atmosphere to remove pollutants from air; and
a high-pressure pump delivering water to the water supply pipe and the high-pressure nozzle, the high-pressure pump being driven by electricity received from the storage battery.

9. The air curtain system according to claim 7, further comprising:
a water supply pipe disposed along the sidewall of the plant wall and supplying water to the plants planted on the plant wall;
a high-pressure nozzle disposed at an upper end of the water supply pipe and spraying water into the atmosphere to remove pollutants from air; and
a high-pressure pump delivering water to the water supply pipe and the high-pressure nozzle, the high-pressure pump being driven by electricity received from the storage battery.
